(19) United States
(12) Patent Application Publication
(10) Pub. No.: US 2021/0190752 A1
(43) Pub. Date: Jun. 24, 2021

(54) MULTI-FUNCTION WATER QUALITY MONITORING DEVICE

(71) Applicant: Industrial Technology Research Institute, Hsin-chu (TW)

(72) Inventors: SZU-JU LI, Changhua County (TW); LI-DUAN TSAI, Hsinchu City (TW); JUNG-HAO WANG, New Taipei City (TW); ZHI-SHENG HUANG, New Taipei City (TW); CHUN-LIN CHENG, New Taipei City (TW)

(21) Appl. No.: 17/126,088

(22) Filed: Dec. 18, 2020

Related U.S. Application Data
(60) Provisional application No. 62/951,008, filed on Dec. 20, 2019.
(60) Provisional application No. 62/951,348, filed on Dec. 20, 2019.

(30) Foreign Application Priority Data
May 26, 2020 (TW) 109206513
Aug. 4, 2020 (TW) 109126368

(51) Int. Cl. G01N 33/18; G01N 27/07; G01N 27/416; G01N 27/36; G01N 27/27

(52) U.S. Cl. CPC G01N 33/1886; G01N 33/1813; G01N 27/07; G01N 27/27; G01N 27/4168; G01N 27/36; G01N 27/4167

(57) ABSTRACT

A multi-function water quality monitoring device is provided, which includes a multi-function water quality monitoring probe and a control module. The multi-function water quality monitoring probe includes a first signal electrode, a first sensing electrode, a second signal electrode and a second sensing electrode. The control module is connected to the probe. When the control module outputs a first time-variant signal to drive the first signal electrode, the first sensing electrode outputs a first water quality signal. When the control module outputs a second time-variant signal to drive the second signal electrode, the first sensing electrode and the second sensing electrode output the first sensing signal and a second sensing signal respectively. When the control module outputs the first time-variant signal and the second time-variant signal to simultaneously drive the first signal electrode and the second signal electrode, the first sensing electrode outputs the first water quality signal.

### CROSS REFERENCE TO RELATED APPLICATION

All related applications are incorporated by reference. The present application is based on, and claims priority from, U.S. Provisional Application No. 62/951,348, filed on Dec. 20, 2019, U.S. Provisional Application No. 62/951,008, filed on Dec. 20, 2019, Taiwan Application No. 109206513, filed on May 26, 2020, and Taiwan Application No. 109126368, filed on Aug. 4, 2020, the disclosures of which are hereby incorporated by reference herein in its entirety.

### TECHNICAL FIELD

The technical field relates to a water quality monitoring device, in particular to a multi-function water quality monitoring device.

### BACKGROUND

Factories in an industrial district would generate a large amount of waste water. However, some factories fail to properly treat waste water, but directly discharge waste water into rivers or other important water bodies, which may pollute domestic water. Thus, it is necessary to frequently monitor domestic water.

The operating principle of a currently available portable water quality monitor is to perform water quality measurement via the electrodes inside the probe thereof. However, the portable water quality monitor can provide only 1-2 water quality parameter measurement functions because being limited by the size thereof. Accordingly, the user cannot obtain more water quality parameters by one measurement operation, so the application of the portable water quality monitor is limited.

The user should frequently replace the probe of the portable water quality monitor in order to measure different water quality parameters, which would waste a lot of time. Therefore, the portable water quality monitor is not convenient in use.

### SUMMARY

An embodiment of the disclosure relates to a multi-function water quality monitoring device, which includes a multi-function water quality monitoring probe and a control module. The multi-function water quality monitoring probe includes a first signal electrode, a first sensing electrode, a second signal electrode and a second sensing electrode. The control module is connected to the multi-function water quality monitoring probe. When the control module outputs a first time-variant signal to drive the first signal electrode, the first sensing electrode outputs a first water quality signal. When the control module outputs a second time-variant signal to drive the second signal electrode, the first sensing electrode and the second sensing electrode output the first sensing signal and a second sensing signal respectively. When the control module outputs the first time-variant signal and the second time-variant signal to simultaneously drive the first signal electrode and the second signal electrode, the first sensing electrode outputs the first water quality signal.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The disclosure will become more fully understood from the detailed description given herein below and the accompanying drawings which are given by way of illustration only, and thus are not limitative of the disclosure and wherein:

FIG. 1 is a system schematic view of a multi-function water quality monitoring device in accordance with a first embodiment of the disclosure.

FIG. 2 is a system schematic view of a multi-function water quality monitoring device in accordance with a second embodiment of the disclosure.

FIG. 3 is a schematic view of a scheduling mechanism of the multi-function water quality monitoring device in accordance with the second embodiment of the disclosure.

FIG. 4 is a first schematic view of an operational process of the multi-function water quality monitoring device in accordance with the second embodiment of the disclosure.

FIG. 5 is a second schematic view of the operational process of the multi-function water quality monitoring device in accordance with the second embodiment of the disclosure.

FIG. 6 is a third schematic view of the operational process of the multi-function water quality monitoring device in accordance with the second embodiment of the disclosure.

FIG. 7 is a stereoscopic view of a multi-function water quality monitoring device in accordance with a third embodiment of the disclosure.

FIG. 8 is a side view of the multi-function water quality monitoring device in accordance with the third embodiment of the disclosure.

FIG. 9A is a first schematic view of the multi-function water quality monitoring device in accordance with the third embodiment of the disclosure.

FIG. 9B is a second schematic view of the multi-function water quality monitoring device in accordance with the third embodiment of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

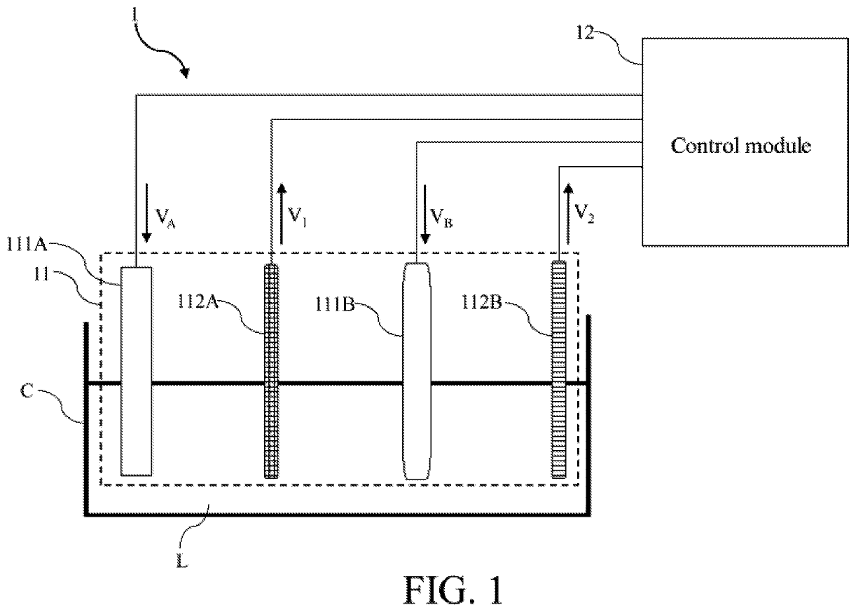

FIG. 1 is a system schematic view of a multi-function water quality monitoring device in accordance with a first embodiment of the disclosure. As shown in FIG. 1, the multi-function water quality monitoring device 1 includes a multi-function monitoring probe 11 and a control module 12.

The multi-function water quality monitoring probe 11 is connected to the control module 12, and includes a first signal electrode 111A, a first sensing electrode 112A, a second signal electrode 111B and a second sensing electrode 112B. When the control module 12 outputs a first time-variant signal V_{A} to the first signal electrode 111A, such as a square wave signal, sinusoidal signal, periodic signal or other time-variant signals. When the control module 12 outputs a second time-variant signal V_{B} to the second signal electrode 111B. For example, the control module 12 outputs a constant voltage to the second signal electrode 111B within a time period and outputs another constant voltage to the second signal electrode 111B within another time period. Besides, the second signal electrode 111B may be also grounded. Moreover, each of the first sensing electrode 112A and the second sensing electrode 112B can generate a voltage signal or a current signal when contacting a liquid sample. In the embodiment, the first signal electrode 111A may be a metal electrode, such as Pt, Au, etc. The signal electrode 111B may be a glass electrode containing electrolyte, such as Ag/AgCl reference electrode, a calomel electrode or an electrode containing conductive material (e.g. Pt, Au, etc.). The first sensing electrode 112A may be an inert metal electrode, such as Pt, Au, etc. The second sensing electrode 112B may be a metal electrode containing ion-selective thin film and electrolyte, such as Ag/AgCl measurement electrode, etc. The second sensing electrode 112B may be also an electrode containing material with high-sensitivity to pH value, such as ITO (Indium Tin Oxide) electrode, etc.

When the multi-function water quality monitoring probe 11 is immersed into the liquid sample L in the container C, the control module 12 outputs the first time-variant signal V_{A} and the second time-variant signal V_{B} to the first signal electrode 111A and the second signal electrode 111B one after another or simultaneously, and then receives the signals from the first sensing electrode 112A and the second sensing electrode 112B. Afterward, the control module 12 calculates several water quality parameters according to the potential difference or current between the first signal electrode 111A, the second signal electrode 111B, the first sensing electrode 112A and the second sensing electrode 112B.

When the control module 12 outputs the first time-variant signal V_{A} to the first signal electrode 111A to drive the first signal electrode 111A, the first sensing electrode 112A outputs a first water quality signal V₁ to the control module 12. Then, the control module 12 calculates a first water quality parameter according to the first time-variant signal V_{A} and the first water quality signal V₁. In the embodiment, the first water quality parameter may be the EC (electrical conductivity) value.

When the control module 12 outputs the first time-variant signal V_{A} to the second signal electrode 111B to drive the second signal electrode 111B, the first sensing electrode 112A and the second sensing electrode 112B output the first water quality signal V₁ and a second water quality signal V₂ to the control module 12 respectively. Then, the control module 12 calculates a second water quality parameter and a third water quality parameter according to the first time-variant signal V_{A}, the first water quality signal V₁ and the second water quality signal V₂. In the embodiment, the second water quality parameter may be the ORP (Oxidation-Reduction Potential) value and the third water quality parameter may be the pH value.

When the control module 12 simultaneously outputs the first time-variant signal V_{A} and the second time-variant signal V_{B} to the first signal electrode 111A and the second signal electrode 111B so as to drive the first signal electrode 111A and the second signal electrode 111B at the same time, the first sensing electrode 112A outputs the first water quality parameter V₁ to the control module 12. However, the control module 12 calculates a fourth water quality parameter according to the first time-variant signal V_{A}, the second time-variant signal V_{B} and the first water quality signal V₁. In the embodiment, the fourth water quality parameter may be the heavy metal concentration value (e.g. Hg-ion, Cd-ion, Cr-ion, Cu-ion, Pb-ion, Zn-ion, etc.).

The multi-function water quality monitoring device 1 may further include a display module; in one embodiment, the display module may be a liquid crystal display or other similar displays. The display module can display the EC value, the ORP value, the pH value and the heavy metal concentration value. In addition, the multi-function water quality monitoring device 1 may further include a wireless transmission module, which may be a Bluetooth module, a Wi-Fi module or other wireless communication modules. Therefore, the control module 12 may transmit the EC value, the ORP value, the pH value and the heavy metal concentration value to an electronic device via the wireless transmission module.

Via the above special switching mechanism and the electrode arrangement, the multi-function water quality monitoring device 1 can provide at least 4 water quality monitoring functions at a time without increasing the number of the electrodes. Besides, the size of the multi-function water quality monitoring device 1 will not increase and the performance thereof can be effectively enhanced.

The embodiment just exemplifies the disclosure and is not intended to limit the scope of the disclosure. Any equivalent modification and variation according to the spirit of the disclosure is to be also included within the scope of the following claims and their equivalents.

It is worthy to point out that the currently available portable water quality monitor can provide only 1-2 water quality parameter measurement functions because being limited by the size thereof. Accordingly, the user cannot obtain more water quality parameters by one measurement operation, so the application of the portable water quality monitor is limited. On the contrary, according to one embodiment of the disclosure, the multi-function water quality monitoring device includes a multi-function water quality monitoring probe, which can provide more than 3 water quality monitoring functions via a special switching mechanism. Accordingly, the performance of the multi-function water quality monitoring device can be significantly enhanced.

Besides, the user should frequently replace the probe of the currently available portable water quality monitor in order to measure different water quality parameters, which would waste a lot of time. Therefore, the portable water quality monitor is not convenient in use. On the contrary, according to one embodiment of the disclosure, the multi-function water quality monitoring device can provide more than 3 water quality monitoring functions, so the user can measure more water quality parameters without replacing the probe of the device, which is more efficient in use.

Moreover, according to one embodiment of the disclosure, the multi-function water quality monitoring device has a special switching mechanism and electrode arrangement, so can provide more than 3 water quality monitoring functions via the special switching mechanism without increasing the size thereof. Thus, the multi-function water quality monitoring device can be a portable device, which is more comprehensive in use.

Furthermore, according to one embodiment of the disclosure, the structure of the multi-function water quality monitoring device is simple, so can achieve the desired technical effects without greatly increasing the cost thereof. Therefore, the multi-function water quality monitoring device is of high commercial value. As described above, the multi-function water quality monitoring device according to the embodiments can actually achieve unpredictable technical effects.

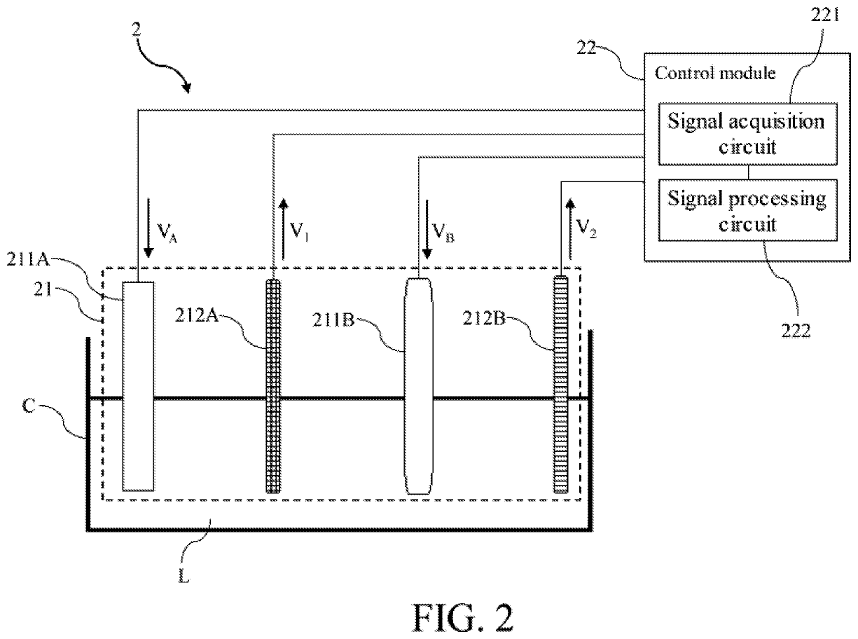

FIG. 2 is a system schematic view of a multi-function water quality monitoring device in accordance with a second embodiment of the disclosure. As shown in FIG. 2, the multi-function water quality monitoring device 2 includes a multi-function monitoring probe 21 and a control module 22.

The multi-function water quality monitoring probe 21 is connected to the control module 22, and includes a first signal electrode 211A, a first sensing electrode 212A, a second signal electrode 211B and a second sensing electrode 212B. The control module 22 outputs a first time-variant signal V_{A} and a second time-variant signal V_{B} to the first signal electrode 211A and the second signal electrode 211B one after another or simultaneously. Similarly, each of the first sensing electrode 212A and the second sensing electrode 212B can generate a voltage signal or a current signal when contacting a liquid sample. In the embodiment, the first signal electrode 211A may be a metal electrode. The signal electrode 211B may be a glass electrode containing electrolyte or an electrode containing conductive material. The first sensing electrode 212A may be an inert metal electrode. The second sensing electrode 212B may be a metal electrode containing ion-selective thin film and electrolyte.

The control module 22 includes a signal acquisition circuit 221 and a signal processing circuit 222. The signal acquisition circuit 221 is connected to the signal processing circuit 222.

When the multi-function water quality monitoring probe 21 is immersed into the liquid sample L in the container C, the signal acquisition circuit 221 outputs the first time-variant signal V_{A} and the second time-variant signal V_{B} to the first signal electrode 211A and the second signal electrode 211B one after another or simultaneously, and then receives the signals from the first sensing electrode 212A and the second sensing electrode 212B. Afterward, the signal processing circuit 222 calculates several water quality parameters according to the potential difference or current between the first signal electrode 211A, the second signal electrode 211B, the first sensing electrode 212A and the second sensing electrode 212B.

When the signal acquisition circuit 221 outputs the first time-variant signal V_{A} to the first signal electrode 221A to drive the first signal electrode 221A, the first sensing electrode 212A outputs a first water quality signal V₁ to the signal acquisition circuit 221. Then, the signal acquisition circuit 221 amplifies the potential difference between the first time-variant signal V_{A} and the first water quality signal V₁, and transmits the potential difference to the signal processing circuit 222. Then, the signal processing circuit 222 calculates the EC value Cₐ (the first water quality parameter) according to the potential difference between the first time-variant signal V_{A} and the first water quality signal V₁. However, if the liquid sample L is a highly concentrated solution, the signal processing circuit 222 calculates the EC value C_{b} according to the first time-variant signal V_{A} and the second water quality signal V₂.

When the signal acquisition circuit 221 outputs the second time-variant signal V_{B} to the second signal electrode 211B to drive the second signal electrode 211B, the first sensing electrode 212A and the second sensing electrode 212B outputs the first water quality signal V₁ and the second water quality signal V₂ to the signal acquisition circuit 221 respectively. Then, the signal acquisition circuit 221 amplifies the potential difference between the second time-variant signal V_{B}, the first water quality signal V₁ and the second water quality signal V₂, and transmits the potential difference to the signal processing circuit 222. After that, the signal processing circuit 222 calculates the ORP value C_{c} (the second water quality parameter) and the pH value C_{d} (the third water quality parameter) according to the potential difference between the second time-variant signal V_{B}, the first water quality signal V₁ and the second water quality signal V₂.

When the signal acquisition circuit 221 outputs the first time-variant signal V_{A} and the second time-variant signal V_{B} to the first signal electrode 211A and the second signal electrode 211B in order to simultaneously drive the first signal electrode 211A and the second signal electrode 211B, the first sensing electrode 212A outputs the first water quality signal V₁ to the signal acquisition circuit 221. Afterward, the signal acquisition circuit 221 amplifies the potential difference between the first time-variant signal V_{A}, second time-variant signal V_{B} and the first water quality signal V₁, and transmits the potential difference to the signal processing circuit 222. After that, the signal processing circuit 222 calculates the Cu-ion concentration value (the fourth water quality parameter) according to the potential difference between the first time-variant signal V_{A}, the second time-variant signal V_{B} and the first water quality signal V₁.

Similarly, the multi-function water quality monitoring device 2 may further include a display module and a wireless transmission module. The display module can display the EC value, the ORP value, the pH value and the heavy metal concentration value. The control module 22 can transmit the EC value, the ORP value, the pH value and the heavy metal concentration value to an electronic device via the wireless transmission module.

Via the above special switching mechanism and electrode arrangement, the multi-function water quality monitoring device 2 can provide 4 different measurement functions, including the EC value, the ORP value, the pH value and the heavy metal concentration value, at a time without increasing the number of the electrodes. The above design would not increase the size of the multi-function water quality monitoring device 2, but can remarkably improve the performance of the multi-function water quality monitoring device 2.

The embodiment just exemplifies the disclosure and is not intended to limit the scope of the disclosure. Any equivalent modification and variation according to the spirit of the disclosure is to be also included within the scope of the following claims and their equivalents.

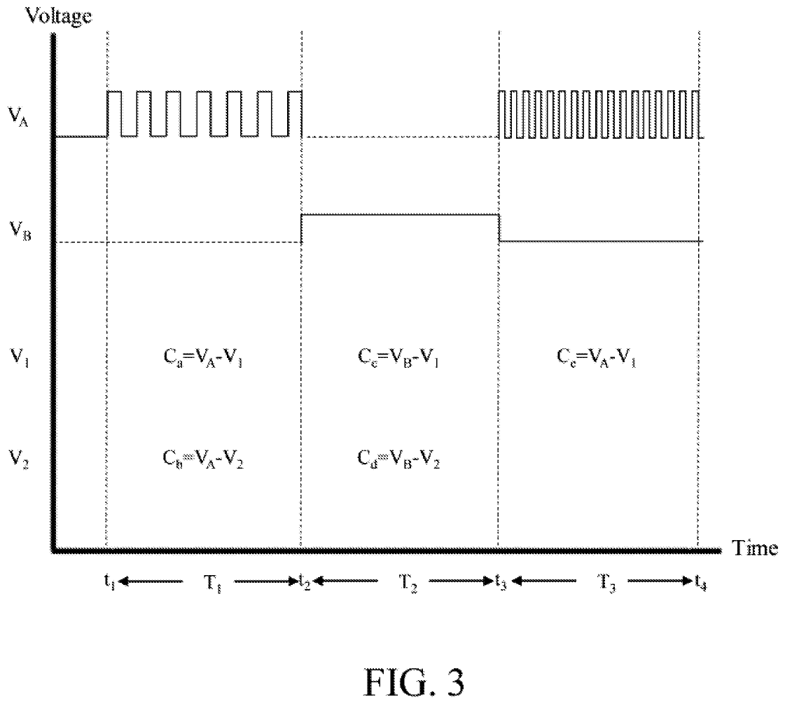

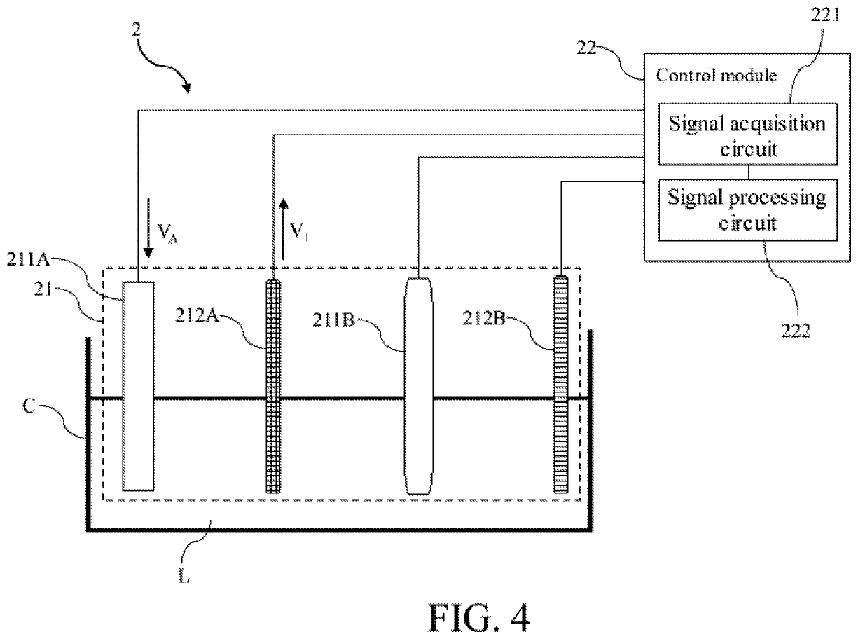

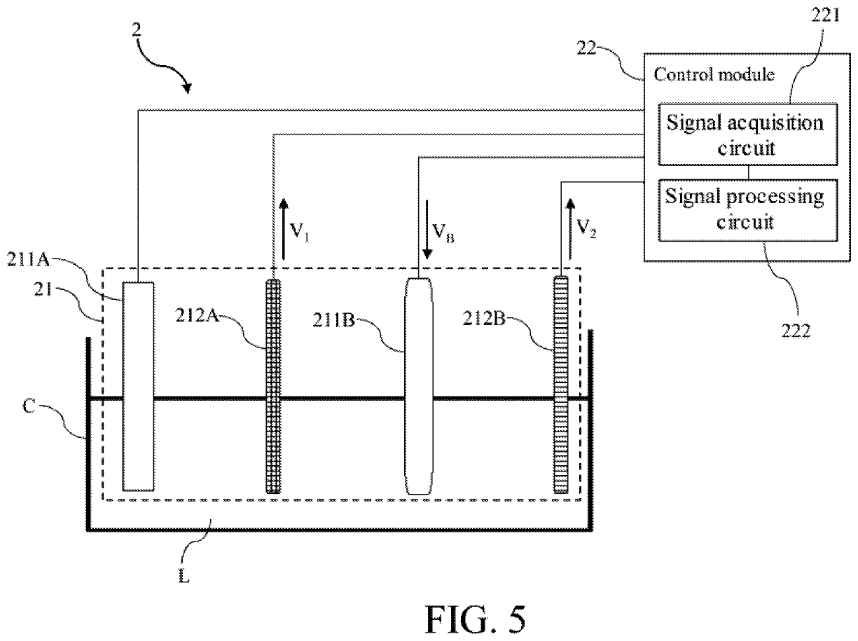

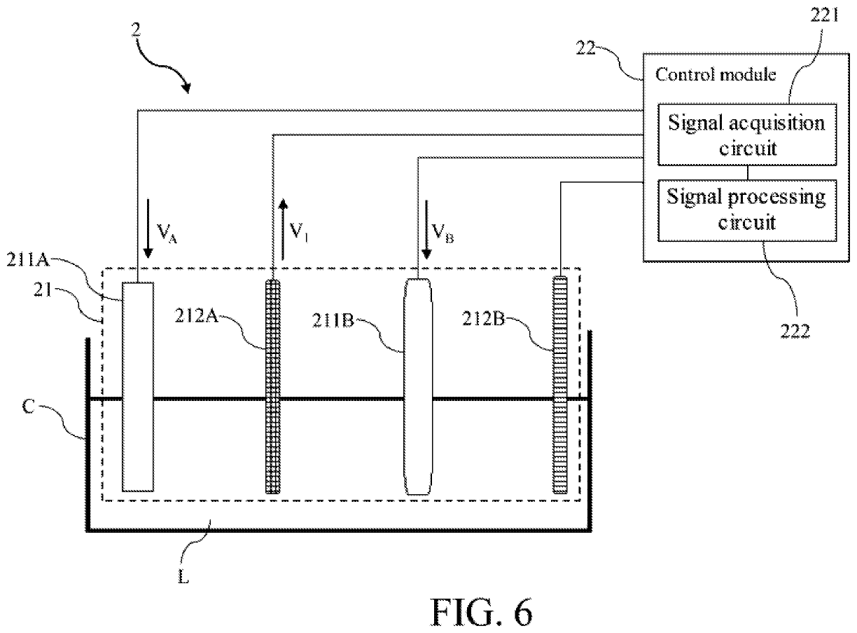

Please refer to FIG. 3, FIG. 4, FIG. 5 and FIG. 6. FIG. 3 is a schematic view of a scheduling mechanism of the multi-function water quality monitoring device in accordance with the second embodiment of the disclosure. FIG. 4, FIG. 5 and FIG. 6 are a first schematic view, a second schematic view and a third schematic view of an operational process of the multi-function water quality monitoring device in accordance with the second embodiment of the disclosure respectively. The multi-function water quality monitoring device 2 of the embodiment can perform a scheduling mechanism in order to measure the EC value, the ORP value, the pH value and the heavy metal concentration value respectively. Then, the multi-function water quality monitoring device 2 of the embodiment can display the EC value, the ORP value, the pH value and the heavy metal concentration value.

As shown in FIG. 3 and FIG. 4, the signal acquisition circuit 221 outputs the first time-variant signal V_{A} to the first signal electrode 211A to drive the first signal electrode 211A between the first time point t₁ and the second time point t₂ (i.e. the first time period T₁). Meanwhile, the signal acquisition circuit 221 amplifies the potential difference between the first time-variant signal V_{A} and the first water quality signal V₁, and transmits the potential difference to the signal processing circuit 222. Next, the signal processing circuit 222 calculates the EC value Cₐ according to the potential difference between the first time-variant signal V_{A} and the first water quality signal V₁, and displays the EC value Cₐ via the display module within the first time period T₁. When the solution is a highly concentrated solution, the signal acquisition circuit 221 amplifies the potential difference between the first time-variant signal V_{A} and the second water quality signal V₂, and transmits the potential difference to the signal processing circuit 222. Next, the signal processing circuit 222 calculates the EC value C_{b} according to the potential difference between the first time-variant signal V_{A} and the second water quality signal V₂, and displays the EC value C_{b} via the display module within the first time period T₁.

As shown in FIG. 3 and FIG. 5, the signal acquisition circuit 221 switches from the first signal electrode 211A to the second signal electrode 211B, outputs the second time-variant signal V_{B} to the second signal electrode 211B to drive the second signal electrode 211B, and receives the first water quality signal V₁ and the second water quality signal V₂ from the first sensing electrode 212A and the second sensing electrode 212B respectively between the second time point t₂ and the third time point t₃ (i.e. the second time period T₂). Meanwhile, the signal acquisition circuit 221 amplifies the potential difference between the second time-variant signal V_{B}, the first water quality signal V₁ and the second water quality signal V₂, and transmits the potential difference to the signal processing circuit 222. Afterward, the signal processing circuit 222 calculates the pH value Cd and the ORP value C_{c} according to the potential difference between the second time-variant signal V_{B}, the first water quality signal V₁ and the second water quality signal V₂, and displays the pH value C_{d} and the ORP value C_{c} via the display module within the second time period T₂.

Finally, the signal acquisition circuit 221 outputs first time-variant signal V_{A} and the second time-variant signal V_{B} to the first signal electrode 211A and the second signal electrode 211B between the third time point t₃ and the fourth time point t₄ (i.e. the third time period T₃) so as to simultaneously drive the first signal electrode 211A and the second signal electrode 211B, and receive the first water quality signal V₁ from the first sensing electrode 212A. In the meanwhile, the signal acquisition circuit 211 amplifies the potential difference between the first time-variant signal V_{A}, the second time-variant signal V_{B} and the first water quality signal V₁, and transmits the potential difference to the signal processing circuit 222. Afterward, the signal processing circuit 222 calculates the Cu-ion concentration value Cₑ according to the potential difference between the first time-variant signal V_{A}, the second time-variant signal V_{B} and the first water quality signal V₁, and displays the Cu-ion concentration value Cₑ within the third time period T₃ via the display module.

As described above, the multi-function water quality monitoring device 2 can provide a special scheduling mechanism to automatically switch the electrodes of the multi-function water quality monitoring probe 21 and can orderly display several water quality parameters via the display module. Thus, the multi-function water quality monitoring device 2 can be more convenient in use.

The embodiment just exemplifies the disclosure and is not intended to limit the scope of the disclosure. Any equivalent modification and variation according to the spirit of the disclosure is to be also included within the scope of the following claims and their equivalents.

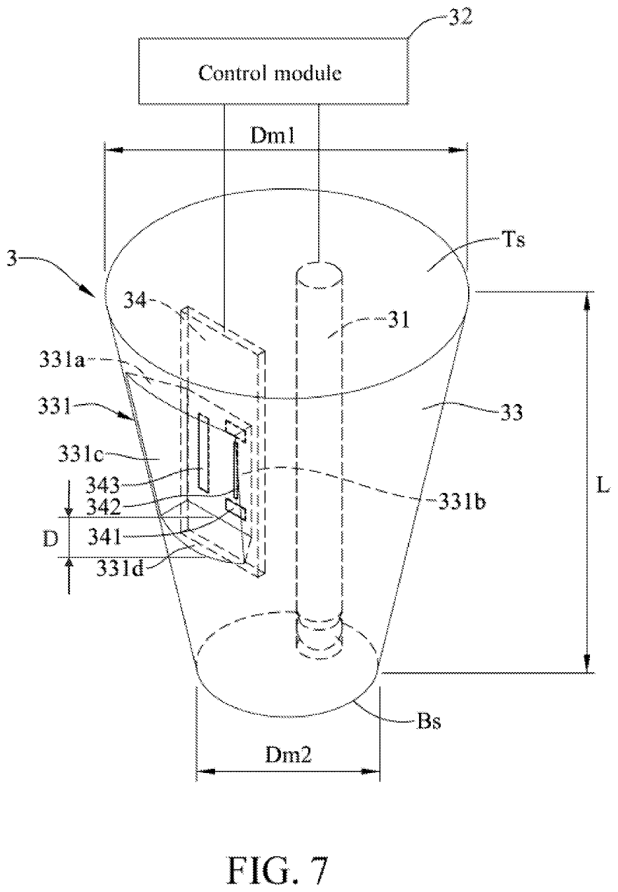

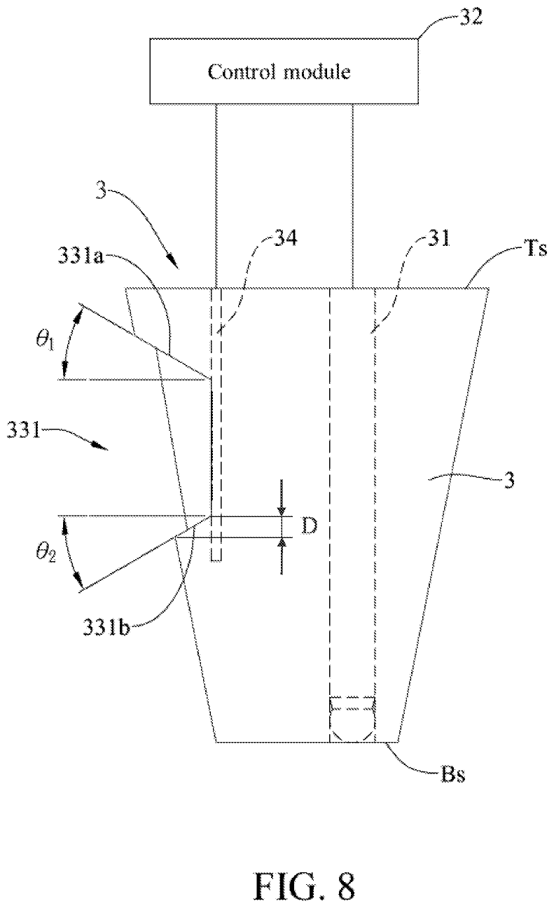

Please refer to FIG. 7 and FIG. 8, which are a stereoscopic view and a side view of a multi-function water quality monitoring device in accordance with a third embodiment of the disclosure respectively. As shown in FIG. 7, the multi-function water quality monitoring device 3 includes a housing 33, a plate sensing element 34, a rod-shaped sensing element 31 and a control module 32.

The housing 33 includes a sensing window 331. The sensing window 331 has an upper wall 331a, a left wall 331b, a right wall 331c and a lower wall 331d. The plate sensing element 34 is disposed at the bottom of the sensing window 331. In this way, the sensing window 331 and the plate sensing element 34 can form a storage space for containing a liquid sample. As shown in FIG. 8, the inclination (i.e. the included angle θ₁ between the upper wall 331a and the horizontal direction H) of the upper wall 331a is less than or equal to 15°. Similarly, the inclination of the left wall 331b and the inclination of the right wall 331c are also less than or equal to 15°. The inclination (i.e. the included angle θ₂ between the lower wall 331d and the horizontal direction H) of the lower wall 331d is 30°˜45°, and the distance D between the top of the lower wall 331d to the bottom thereof is 5˜7.5 mm.

As shown in FIG. 7, the plate sensing element 34 is disposed at the bottom of the sensing window 331, and includes a first signal electrode 341, a temperature sensor 342 and a first sensing electrode 343. The first signal electrode 341, the temperature sensor 342 and the first sensing electrode 343 are disposed in the sensing window 331. The first signal electrode 341, the temperature sensor 342 and the first sensing electrode 343 are corresponding to different water quality parameters, such as electrical conductivity (EC), pH, etc. The plate sensing element 34 can be manufactured by thin-film process or screen printing technology in order to integrate several different signal electrodes and sensing electrodes with one another; the plate sensing element 34 has many advantages, such as small size, easy to maintain, low cost, etc. In this embodiment, the first signal electrode 341 and first sensing electrode 343 may be applicable to, but not limited to, electrical conductivity measurement. In another embodiment, the first signal electrode 341 and first sensing electrode 343 may further include various electrochemical sensors. The first signal electrode 341 and first sensing electrode 343 generate sensing signals corresponding to the water quality parameters thereof respectively when contacting the liquid sample in the sensing window 331. The functions and operational process of the plate sensing element 34 (the first signal electrode 341 and the first sensing electrode 343) are already described in the first embodiment and the second embodiment, so would not be described herein again.

The rod-shaped sensing element 31 is disposed in the housing 33. The rod-shaped sensing element 31 extends from the top Ts of the housing 33 to the bottom Bs of the housing 33, and protrudes from the top Ts of the housing 33 to the bottom Bs of the housing 33 respectively. In this embodiment, the diameter Dm1 of the top Ts of the housing 33 is about 40 mm; the diameter of the bottom Bs of the housing 33 is about 20 mm; the height L of the housing 33 is about 50 mm. The length of the rod-shaped sensing element 31 is substantially equal to the height L of the housing 33. The above structure is just for illustration; the sizes of the above elements can be adjusted according to actual requirements.

Similarly, the rod-shaped sensing element 31 also include a second signal electrode and a second sensing electrode. The functions and operational process of the rod-shaped sensing element 31 are already described in the first embodiment and the second embodiment, so would not be described herein again.

As set forth above, the multi-function water quality monitoring device 3 can further integrate different signal electrodes and sensing electrodes with one another sensors via the plate sensing element 34 and the rod-shaped sensing element 31, so can detect different water quality parameters via the sensing window 331 and the bottom Bs, protruding from the housing 33, of the rod-shaped sensing element 31, which is more flexible in use.

As described above, the multi-function water quality monitoring device 3 has a sensing window 331 having a special structure design. Thus, when the multi-function water quality monitoring device 3 is placed to be parallel to the horizontal direction, the sensing window 331 can be filled with a liquid sample and can prevent the liquid sample from flowing out of the sensing window 331. When the multi-function water quality monitoring device 3 is placed to be parallel to the vertical direction, the liquid sample can completely flow out of the sensing window 331 in a short time. Accordingly, the multi-function water quality monitoring device 3 can achieve great practicality.

Moreover, the multi-function water quality monitoring device 3 may also have a control module having several buttons and a display screen with a view to serve as a portable device. In this way, the user can operate the multi-function water quality monitoring device 3 via the control module to monitor the water quality of a liquid sample and obtain the sensing results via the display screen of the control module, which is more convenient in use.

The embodiment just exemplifies the disclosure and is not intended to limit the scope of the disclosure. Any equivalent modification and variation according to the spirit of the disclosure is to be also included within the scope of the following claims and their equivalents.

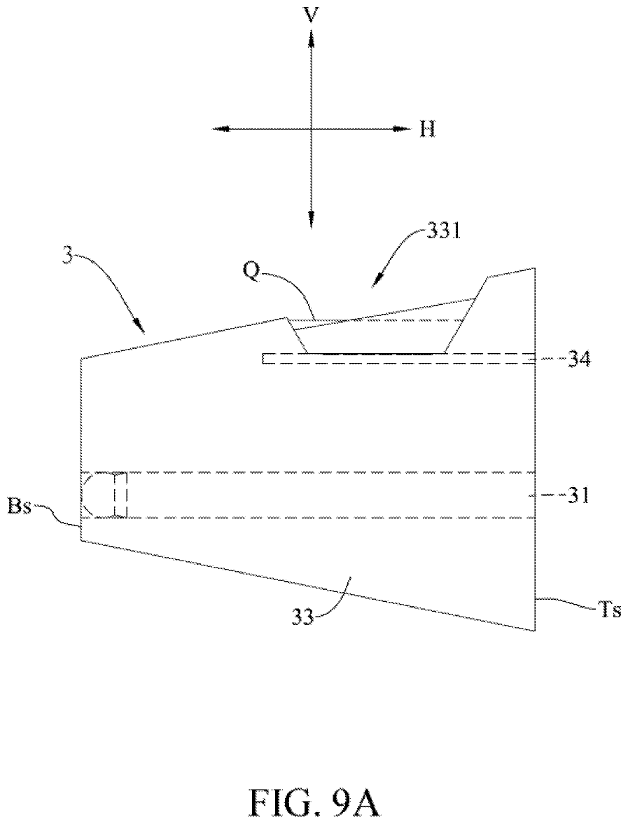

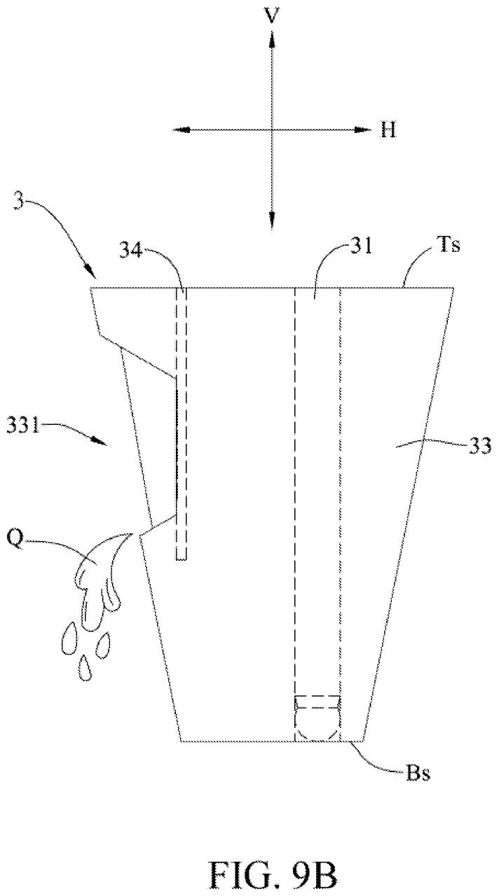

Please refer to FIG. 9A and FIG. 9B, which are a first schematic view and a second schematic view of the multi-function water quality monitoring device in accordance with the third embodiment of the disclosure respectively. Please also refer to FIG. 7. As shown in FIG. 7, the sensing window 331 of the housing 33 of the multi-function water quality monitoring device 3 has the upper wall 331a, the left wall 331b, the right wall 331c and the lower wall 331d connected to each other. The inclination of the upper wall 331a, the inclination of the left wall 331b and the inclination of the right wall 331c are less than or equal to 15°. The inclination of the lower wall 331d is 30° ˜′45°. The distance D between the top of the lower wall 331d to the bottom thereof is 5˜7.5 mm. As shown in FIG. 9A, via the above structure design, when the multi-function water quality monitoring device 3 is placed to be parallel to the horizontal direction H (i.e. the sensing window 331 is parallel to the horizontal direction H), the liquid sample Q would not flow out of the sensing window 331. As shown in FIG. 9B, when the multi-function water quality monitoring device 3 is placed to be parallel to the vertical direction V (i.e. the sensing window 331 is parallel to the vertical direction V), the special structure design of the sensing window 331 can make the liquid sample Q completely flow out of the sensing window 331 in a short time. In this way, the multi-function water quality monitoring device 3 can swiftly and efficiently detect the water quality of the liquid sample Q, so can achieve great practicality.

To sum up, according to one embodiment of the disclosure, the multi-function water quality monitoring device includes a multi-function water quality monitoring probe, which can provide more than 3 water quality monitoring functions via a special switching mechanism. Accordingly, the performance of the multi-function water quality monitoring device can be significantly enhanced.

According to one embodiment of the disclosure, the multi-function water quality monitoring device can provide more than 3 water quality monitoring functions, so the user can measure more water quality parameters without replacing the probe of the device, which is more efficient in use.

According to one embodiment of the disclosure, the multi-function water quality monitoring device can provide a special scheduling mechanism to automatically switch the electrodes of the multi-function water quality monitoring probe and can orderly display several water quality parameters via the display module. Thus, the multi-function water quality monitoring device can be more convenient in use.

Besides, according to one embodiment of the disclosure, the multi-function water quality monitoring device has a special switching mechanism and electrode arrangement, so can provide more than 3 water quality monitoring functions via the special switching mechanism without increasing the size thereof. Thus, the multi-function water quality monitoring device can be a portable device, which is more comprehensive in use.

Further, according to one embodiment of the disclosure, the multi-function water quality monitoring device can integrate several sensors with different functions via a sensing window, so can detect several water quality parameters via the sensing window. Accordingly, the multi-function water quality monitoring device can be more flexible in use.

Moreover, according to one embodiment of the present disclosure, the multi-function water quality monitoring device has a sensing window having a special structure design. Thus, when the multi-function water quality monitoring device is placed to be parallel to the horizontal direction, the sensing window can be filled with a liquid sample and can prevent the liquid sample from flowing out of the sensing window. When the multi-function water quality monitoring device is placed to be parallel to the vertical direction, the liquid sample can completely flow out of the sensing window in a short time. Accordingly, the multi-function water quality monitoring device can achieve great practicality.

Furthermore, according to one embodiment of the disclosure, the structure of the multi-function water quality monitoring device is simple, so can achieve the desired technical effects without greatly increasing the cost thereof. Therefore, the multi-function water quality monitoring device is of high commercial value.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A multi-function water quality monitoring device, comprising:
a multi-function water quality monitoring probe, comprising a first signal electrode, a first sensing electrode, a second signal electrode and a second sensing electrode; and
a control module, connected to the multi-function water quality monitoring probe;
wherein when the control module outputs a first time-variant signal to drive the first signal electrode, the first sensing electrode outputs a first water quality signal, wherein when the control module outputs a second time-variant signal to drive the second signal electrode, the first sensing electrode and the second sensing electrode output the first sensing signal and a second sensing signal respectively, wherein when the control module outputs the first time-variant signal and the second time-variant signal to simultaneously drive the first signal electrode and the second signal electrode, the first sensing electrode outputs the first water quality signal.

2. The multi-function water quality monitoring device of claim 1, wherein the control module calculates a first water quality parameter according to the first time-variant signal, and calculates a second water quality parameter and a third water quality parameter according to the second time-variant signal, the first water quality signal and the second water quality signal, and calculates a fourth water quality parameter according to the first time-variant signal, the second time-variant signal and the first water quality signal.

3. The multi-function water quality monitoring device of claim 2, wherein the first water quality parameter is an electrical conductivity value, the second water quality parameter is an oxidation-reduction potential value, the third water quality parameter is a pH value and the fourth water quality parameter is a heavy metal concentration value.

4. The multi-function water quality monitoring device of claim 3, further comprising a display module, wherein the display module displays the electrical conductivity value, the oxidation-reduction potential value, the pH value and the heavy metal concentration value.

5. The multi-function water quality monitoring device of claim 1, wherein the control module comprises a signal acquisition circuit, wherein the signal acquisition circuit amplifies a plurality of potential difference between the first time-variant signal, the second time-variant signal, the first water quality signal and the second water quality signal.

6. The multi-function water quality monitoring device of claim 5, wherein the signal acquisition circuit switches between the first signal electrode and the second signal electrode.

7. The multi-function water quality monitoring device of claim 5, wherein the control module further comprises a signal processing circuit, and the signal processing circuit calculates a first water quality parameter according to the first time-variant signal and the first water quality signal, and calculates a second water quality parameter and a third water quality parameter according to the second time-variant signal, the first water quality signal and the second water quality signal, and calculates a fourth water quality parameter according to the first time-variant signal, the second time-variant signal and the first water quality signal.

8. The multi-function water quality monitoring device of claim 1, wherein the first time-variant signal is a square wave signal, a sinusoidal wave signal or a periodic signal.

9. The multi-function water quality monitoring device of claim 1, wherein the second signal electrode is grounded.

10. The multi-function water quality monitoring device of claim 1, wherein the first signal electrode is a metal electrode.

11. The multi-function water quality monitoring device of claim 1, wherein the second signal electrode is a glass electrode containing electrolyte or an electrode containing conductive material.

12. The multi-function water quality monitoring device of claim 1, wherein the first sensing electrode is an inert metal electrode.

13. The multi-function water quality monitoring device of claim 1, wherein the second sensing electrode is a metal electrode containing ion-selective thin film and electrolyte.

14. The multi-function water quality monitoring device of claim 1, wherein the control module executes a scheduling mechanism in order to drive the first signal electrode within a first time period, drives the second signal electrode within a second time period, and simultaneously drives the first signal electrode and the second signal electrode within a third time period.

15. The multi-function water quality monitoring device of claim 1, further comprising:
a housing, comprising a sensing window having an upper wall, a left wall, a right wall and a lower wall connected to each other, wherein an inclination of the upper wall, an inclination of the left wall and an inclination of the right wall are less than or equal to 15°, an inclination of the lower wall is 30°˜45°, and the control module is disposed in the housing;
a plate sensing element, connected to the control module and disposed at a bottom of the sensing window, wherein the plate sensing element comprises the first signal electrode and the first sensing electrode disposed in the sensing window, wherein the first signal electrode and the first sensing electrode generates a sensing signal corresponding to a water quality parameter when the first signal electrode and the first sensing electrode contact a liquid sample; and
a rod-shaped sensing element, connected to the control module and disposed in the housing, wherein the rod-shaped sensing element comprises the second signal electrode and the second sensing electrode, wherein the second signal electrode and the second sensing electrode generates another sensing signal corresponding to another water quality parameter when the second signal electrode and the second sensing electrode contact the liquid sample;
wherein the control module generates a plurality of sensing results corresponding to the water quality parameters according to the first signal electrode, the first sensing electrode, the second signal electrode and the second sensing electrode.

16. The multi-function water quality monitoring device of claim 15, wherein a distance between a top of the lower wall and a bottom of the lower wall is 5˜7.5 mm.

17. The multi-function water quality monitoring device of claim 15, wherein the rod-shaped sensing element extends from a top of the housing to a bottom of the housing.
